(19) United States
(12) Patent Application Publication
(10) Pub. No.: US 2003/0220212 A1
(43) Pub. Date: Nov. 27, 2003

(54) Reinforced bar soap

(76) Inventor: Louis DeVitis, Perrineville, NJ (US)

Correspondence Address: Patrick H. Higgins, Mathews, Collins, Shepherd & McKay, Suite 306, 100 Thanet Circle, Princeton, NJ, 08540, US

(21) Appl. No.: 10/404,754

(22) Filed: Apr. 1, 2003

Related U.S. Application Data
(60) Provisional application No. 60/369,171, filed on Apr. 2, 2002.

(51) Int. Cl. A61K 7/50

(52) U.S. Cl. 510/141; 510/144; 510/140

(57) ABSTRACT

A reinforced bar soap is provided to prolong the usage of conventional bar soap and reduce consumption which comprises a reinforcement member in an interior portion thereof. The reinforcement member is preferably at a core position in the interior portion of the bar. Embodiments are provided wherein the reinforcement member is flexible. Reinforced bar soaps are provided, for example, which comprise at least one mesh-type reinforcement member.

### FIELD OF THE INVENTION

The present invention relates to reinforced bar soap for providing longer usage of the soap and hence reduced consumption.

### DESCRIPTION OF THE PRIOR ART

Conventional soaps are produced in bar form with no consideration to wear or breakage. It is desirable to provide a bar soap with improved wear and usability.

### SUMMARY OF THE INVENTION

The present invention comprises a bar of soap including a reinforcement member in an interior portion thereof. The reinforcement member can be formed of a mesh material. The reinforcement member holds the bar soap together as the bar soap is reduced in size during use. Accordingly, the invention eliminates normal soap breakage and produces a longer soap life for usage. The invention will be more fully described by reference to the following written description as well as drawings.

### BRIEF DESCRIPTION OF THE DRAWINGS

FIG. 1 is a perspective view of a reinforced bar soap in accordance with the present invention. The reinforcement member is shown in broken line form in the soap interior.

FIG. 2 is a side view of the reinforced bar soap of the present invention.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

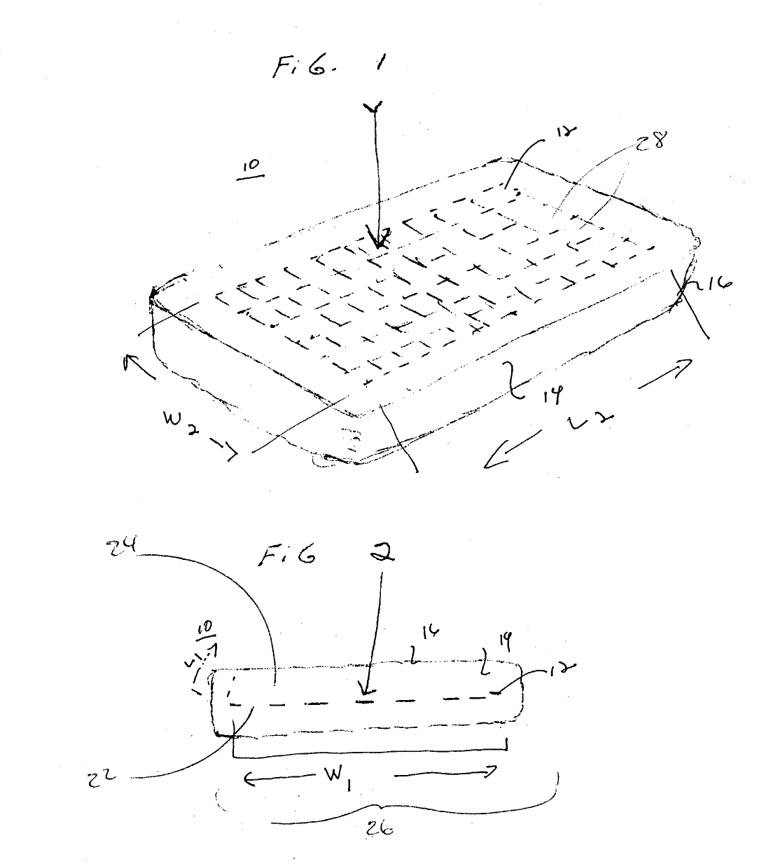

FIG. 1 illustrates a perspective view of reinforced bar soap 10 in accordance with the teachings of the present invention. Reinforcement member 12 is positioned in interior 14 of bar soap 16. Reinforcement member 12 is formed of an open weave or mesh material. Reinforcement member 12 can be formed of a soft material. For example, reinforcement member can be formed of a non-absorbent material such as nylon or flexible plastic. Reinforced bar soap 10 embodiments of the present invention comprise reinforcement members 12 that may, for example, in addition to providing structural support, provide a desirable texture for massaging or scrubbing. Reinforced bar soaps 10 of the present invention are preferred wherein the reinforcement member 12 is flexible. Embodiments of this type for example may be wherein the reinforcement member 12 is formed of an open weave or mesh material. Embodiments are also contemplated wherein the reinforcement member 12 is formed of nylon or plastic. However, any material may be used to construct reinforcement members 12 for use in the current invention, including natural and synthetic materials, provided the material is non-toxic. Natural fibers, sponges, as well as wood may be employed to construct reinforcement members 12. Polymers and other synthetic materials may also be employed to construct reinforcement members 12. A reinforced bar soap 10 is also contemplated wherein the reinforcement member 12 is substantially flat (essentially two dimensional).

As shown in FIG. 2, reinforcement member 12 has a length L₁ and width W₂ which is smaller than length L₂ and width W₂ of bar soap 16 for allowing bar soap 16 to wear down to where reinforcement member 12 is needed, thereby eliminating any protruding mesh. Reinforcement member 12 can be inserted into bar soap 16 manufacturing of bar soap 16. The present invention allows the bar of soap when getting thin, to break and be held together by the reinforcement member such that bar soap 16 can still be used even to point of the soft reinforcement member protruding from the bar soap, thereby producing a much longer life of the bar soap.

Preferred embodiments of reinforced bar soap according to the present invention comprise a reinforcement member in an interior portion of the bar of soap. The reinforcement member, interior to the otherwise substantially conventional bar of soap, provides structural integrity, and enables prolonged use, particularly when the soap wears thin and otherwise would be subject to breaking up in the shower, for example, or the bathtub. In one embodiment a reinforcement member 12 is at a core position 22 in the interior portion 24 of the bar. As used herein core position 22 refers to a position which is substantially at the symmetrical center of the 3-dimensional (3D) body of the bar. In other words, upon normal wear of the bar soap 16, the reinforcement member 12 generally remains substantially in the middle of the soap bar 16 covered by soap. In another embodiment the reinforcement member 12 substantially emulates the shape of the soap bar 16, but remains in the center of the bar throughout the life of the bar. In this type embodiment the reinforcement member helps increase the release of the soap from the bar when the bar becomes worn down in normal use. Particularly, as the exterior surface area 26 of the bar is gradually reduced upon usage, the reinforcement member gradually becomes exposed wherein a plurality of wells 28 are formed during use, thus increasing the surface area of the soap. See. FIG. 1. Embodiments of this type are preferred wherein the reinforcement member substantially emulates the shape of the bar; however, particularly reinforcement members 12 may be more thin than an actual scaled proportion with reference to the shape of the bar. Reinforcement membersl2, particularly mesh-type reinforcement members, are contemplated, for example, that range from about 1/32 inch to about 1/4 inch in thickness.

A method of manufacturing a reinforced bar soap according to the present invention comprises positioning a reinforcement member within a mold for casting the bar soap. This method may yield reinforced bar soap that comprises a central portion, for example, of the reinforcement member extending to one end of the bar.

A container comprising a reinforced bar soap of the present invention is a further aspect of the present invention. Preferred containers are paper packaging, preferably labels, wrapped around the reinforced bar soap of the present invention.

It is to be understood that the above-described embodiments are illustrative of only a few of the many possible specific embodiments which can represent application of the principles of the invention. Numerous and varied other arrangements can be readily devised in accordance with these principles by those skilled in the art without departing from the spirit and scope of the invention.

What is claimed is

1. A reinforced bar soap comprising a reinforcement member in an interior portion thereof.

2. A reinforced bar soap according to claim 1 wherein the reinforcement member is at a core position in the interior portion.

3. A reinforced bar soap according to claim 2 wherein the reinforcement member substantially emulates a shape of the bar.

4. A reinforced bar soap according to claim 2 wherein the reinforcement member is substantially flat.

5. A reinforced bar soap according to claim 2 wherein the reinforcement member is flexible.

6. A reinforced bar soap according to claim 4 wherein the reinforcement member is flexible.

7. A reinforced bar soap according to claim 5 wherein the reinforcement member is formed of nylon or plastic.

8. A reinforced bar soap according to claim 6 wherein the reinforcement member is formed of an open weave or mesh material.

9. A reinforced bar soap according to claim 2 wherein the reinforcement member is formed of an open weave or mesh material.

10. A reinforced bar soap according to claim 2 wherein the reinforcement member is formed from natural fiber, sponge, or wood.

11. A method of manufacturing a reinforced bar soap according to claim 1 comprising positioning a reinforcement member within a mold for casting the bar soap.

12. A container comprising a reinforced bar soap according to claim 1.

13. A container according to claim 12 which is paper packaging wrapped around the reinforced bar soap.

14. A container according to claim 13 which is a label.
